(19) United States
(12) Patent Application Publication
(10) Pub. No.: US 2009/0053810 A1
(43) Pub. Date: Feb. 26, 2009

(54) System and Method for Cultivating Cells

(75) Inventors: Laura Sander, Molndal (SE); Annika Sundberg, Loddekopinge (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/911,233

(22) PCT Filed: Apr. 10, 2006
(86) PCT No.: PCT/SE2006/000424

§ 371 Date: Apr. 4, 2008

(30) Foreign Application Priority Data
Apr. 12, 2005 (SE) 0500817-2

(51) Int. Cl. C12N 5/06; C12M 1/00; F03B 17/00

(52) U.S. Cl. 435/380; 435/303.1; 435/383; 137/561 R

(57) ABSTRACT

The invention relates to a method of introducing a substance into a multi-layered cell factory. A substance is poured into a substance transferring device e.g. a funnel and a tube connected to the cell factory, which directly transfers the substance into the cell factory arranged with its layers extending substantially vertically. The invention also relates to a cell-cultivation system.

### TECHNICAL FIELD

The present invention relates to a method of introducing a substance into a multi-layered cell factory. The invention also relates to a cell cultivation system which comprises a multi-layered cell factory.

### BACKGROUND OF THE INVENTION

Multi-layered cell factories are widely used as part of cell work in laboratories. Cell factories are available from different suppliers, such as Nunc A/S, Roskilde, Denmark and Corning Incorporated Life Sciences, Acton, Mass. 01720, USA. The cell factories are available in different sizes with different numbers of layers which are suitable for different applications. For instance, a 10-stack (ten-layered) version of these factories has been found particularly useful since a large number of cells can be produced with relatively little extra work, equipment or space. There are, however, some problems with current standard methods for filling these cell factories that result in high cost in time and effort and/or result in unsatisfactory filling and harvesting, which means a lower yield of cells than might otherwise be possible.

Even though the current standard method could be improved for cell factories having different numbers of layers, a basic seeding and harvesting procedure will now only be described for a ten-layered cell factory for the sake of simplicity. The numbers given should be regarded as suitable examples.

To seed cells into a ten-layered cell factory, a 1500 ml cell suspension using the appropriate media with the desired cell/ml concentration is introduced into the cell factory. Cells should be in a completely homogeneous suspension and go evenly into all of the layers at roughly the same time in order to avoid different number of cells in different layers.

The cells to be seeded in the cell factory are initially grown in tissue culture flasks. When the cells are harvested from the tissue culture flasks they are suspended in a suitable media. The cell and media (the cell suspension) are transferred to small bottles. For a ten-layered cell factory, three bottles with 500 ml cell suspension in each bottle are prepared generally from the tissue culture flasks. The three bottles will each contain substantially the same number of cells. From the three bottles, the cell suspension is transferred to an intermediate container where the cell suspension is kept until it is introduced to the cell factory for seeding the cells. The intermediate container may be in the form of a large bottle according to one prior art method or in the form of a pipette according to another prior art method, which will be described below in more detail.

In order to harvest cells, media is first removed from the cell factory, while the cells remain attached to the bottom sheet of each layer. Next, 200-500 ml of a rinsing substance, such as Phosphate Buffered Saline (PBS), is introduced to the cell-factory to wash residual media from the cells. The rinsing substance is then removed and 100-150 ml of a dissociation enzyme such as Accutase™ or Trypsin is introduced to the layers of the cell factory and the cell factory is incubated at 37° C. for a few minutes. When the cells have detached, 200-400 ml of media should be introduced into the cell factory in order to stop the enzyme action. This function of stopping enzymatic reactions is one reason for the above described use of the rinsing substance for washing the cells from residual media. If no rinsing substance would be used the dissociation enzyme might not work effectively.

There are two prior art methods currently used to seed and harvest 10-stack cell factories. The first method is illustrated in accompanying FIG. 1 of the drawings. According to this first method, a large bottle 2 contains all the cell suspension which is to be introduced into the multi-layered (e.g. ten-layered) cell factory 4. The cell suspension is introduced into the cell factory 4 via a tube 6 connected to a bottom portion of the large bottle 2. A clamp 7 controls the flow of the cell suspension. In order to have an effective flow of the cell suspension through the tube 6, the large bottle 2 is placed on a large block 8 so that the gravitational force will cause the cell suspension to flow from the large bottle to the cell factory 4 when the clamp is open. The ten layers 10a-10j of the cell factory 4 are interconnected by means of channels. An advantage of this prior art method is that the cell factory 4 may be placed with the layers 10a-10j standing vertically as shown in FIG. 1. This enables the layers 10a-10j to be filled substantially simultaneously, since the interconnecting channels will quickly spread the cell suspension from one vertically standing layer to the next vertically standing neighbouring layer. However, there are some drawbacks with this method. The cells must be handled with care, so for sterility reasons the large bottle 2 must be kept inside a Laminar Air Flow (LAF) bench when it is filled with the cell suspension. Also, since the large and heavy bottle 2 needs the block 8 for holding the large bottle containing the cell suspension, the height of the large bottle 2 in relation to the cell factory 4 is not adjustable. Thus, on the one hand the large bottle 2 must be lifted high enough for the bottom portion thereof to be located at a satisfactory level for filling the cell factory 4 with the aid of gravitation, and on the other hand the higher the large bottle 2 is located the more awkward is the filling of the bottle for the operator, who will have to struggle to get his arm under the hood of the LAF bench in order to be able to introduce the cell suspension into the large bottle 2. Even filling the large bottle 2 before putting it onto the block 8 is quite difficult and tiring for the operator. Furthermore, the large block 8 and bottle 2, due to their bulkiness, may compromise the air flow in the LAF bench. It is also quite difficult to swirl the cell suspension in the large bottle 2 to obtain homogeneity. Another drawback with the method is that it requires autoclaving the entire large bottle 2 with the attached tube 6. Yet another drawback with this method is that it does not easily allow for the subsequent addition of rinsing substance or enzyme.

A second method, which has grown popular, is to use a pipette for introducing the cell suspension into the cell factory. While it may be easier to handle a comparatively small pipette than a large bottle, this second method also has some drawbacks. To fill a ten-layered cell factory with 1500 ml of cell suspension requires filling and emptying a 50 ml pipette 30 times or a 100 ml pipette 15 times (larger pipettes would be difficult to handle). Such pipetting is not only time-consuming but also physically exhausting. The filling can typically take up to 20 minutes. Because of the way the cell factories work, taking that much time to seed a cell factory can result in a very uneven distribution of cells in the layers. This happens because in order to pipet in the cell suspension, the cell factory must be flat on its back with the filling holes up, i.e. perpendicular to the orientation of the first method shown in FIG. 1. This means that pipetting into the cell factory with horizontally aligned layers according to the second method will result in the cell suspension going straight to the bottom layer and only very slowly beginning to fill the other layers after some time, with the top layers receiving little if any cells. In the time it takes to pipet in the whole cell suspension (cells and media), the cells already start to settle to the bottom, thus causing uneven layering of cells in the factory.

### SUMMARY OF THE INVENTION

An object of the present invention is to alleviate the drawbacks of the prior art methods. Another object of the present invention is to accomplish an easy and quick seeding and harvesting procedure in a multi-layered cell factory. These and other objects, which will become apparent in the following, are achieved by a method, a cell-cultivation system and a use as defined by the independent claims.

The present invention is based on the insight that a more efficient handling and introduction of a substance to a multi-layered cell factory may be achieved by allowing the exclusion of an intermediate container, such as the large bottle according to the first described prior art method or the pipette according to the second described prior art method. In other words, the invention allows a substance to be directly introduced into a multi-layered cell factory without the need for an intermediate container to keep the substance until it is to be delivered into the cell factory.

The invention is also based on the insight that by redirecting a fluid communication inlet interface of a multi-layered cell factory having its layers standing vertically, it is possible to obtain a quick and even filling of the layers of the cell factory. Thus, by redirecting a fluid communication inlet interface of the multi-layered cell factory to face upwardly when the cell factory is in the vertically standing position, it allows for pouring the cell suspension or other substances into the cell factory. A pouring action may, contrary to the prior art methods, be quicker and allow easier handling.

According to a first aspect of the invention, a method of introducing a substance into a multi-layered cell factory is provided. The method comprises connecting a substance transferring device to an inlet of the cell factory, arranging the cell factory so that the layers of the cell factory extend substantially vertically, wherein said arranging is performed before, after or simultaneously with connecting the substance transferring device to the inlet of the cell factory, and pouring the substance from an independent container through the substance transferring device which directly transfers the substance into the cell factory.

Thus, the first aspect of the method allows for taking advantage of the vertically extending layers for an even filling of the layers and combines it with pouring the substance directly into the cell factory via a substance transferring device. In other words the substance may be delivered from the independent container into the cell factory in a continuous flow without being stopped. The prior art intermediate substance holding container is thereby avoided and a quick filling is enabled. The independent container should be regarded as a stand-alone container which does not have to be structurally connected to the cell factory or the substance transferring device, but which may if desired stay free from contact with any of the other system components. This is advantageous from a handling and sterility point of view.

The independent container may be any type of vessel, flask, bottle or other means from which the substance is poured. For instance, in case of the substance being a cell suspension containing cells to be seeded in the cell factory, the independent container may be a small bottle into which harvested cells has been transferred from tissue culture flasks, as previously exemplified under the heading “Background of the invention”. In case of the substance being a rinsing substance or a suspended dissociation enzyme, the independent container may be the bottle in which the solution or suspension is provided by the supplier.

The act of pouring not only allows for quicker filling but is also less physically exhausting for the operator. It is more convenient to handle small bottles than an awkward large bottle connected to a tube. Also, rather than the physically exhausting repeated pipetting from a bottle, introducing a risk of contamination due to the repeated pipetting, a single act of pouring from such a bottle is less exhausting and may be safer from a sterility point of view. Also, the total time required for either seeding into or harvesting cells from a factory can have an impact on overall cell health. Good cell viability is often dependent on the cells being returned to their home environment (e.g. a humidified 37° C. incubator) as quickly as possible. The present invention enables a significantly faster handling of cells in factories than the above described prior arts.

In this application, in case of doubt, by vertically extending layers is hereby meant that the layers are standing next to each other with all the layers having substantially the same distance to the “ground level” on which the cell factory is placed. In contrast to vertically extending layers, horizontally extending layers would be located over each other and each one with different spacing to the ground level.

As previously mentioned, the present invention is based on an insight of the possibility to redirect a fluid communication inlet interface of the multi-layered cell factory. This is envisaged in a second aspect of the invention. According to the second aspect of the invention, a method is provided for introducing a substance into a multi-layered cell factory that has an inlet for receiving the substance. The method comprises connecting to the inlet of the cell factory a substance transferring device which comprises its own inlet for receiving the substance and directly transferring the substance to the inlet of the cell factory. The method also comprises pouring the substance into the inlet of the substance transferring device when the opening area of the inlet of the substance transferring device faces another direction than the opening area of the inlet of the cell factory. Advantageously, the inlet of the substance transferring device may be movable relative to the inlet of the cell factory. In other words, if the substance transferring device has a first end connected to the inlet of the cell factory and a second end representing the inlet of the substance transferring device, the second end may be movable relative to the first end for enabling the first end to be arranged at a non-zero angle relative to the second end.

It should be noted that the method according to this aspect is also suitable for and encompasses the idea of introducing the substance into vertically extending layers in accordance with the first aspect of the invention. Also, the pouring of the substance performed in accordance with the second aspect of the invention may suitably be performed from an independent container as explained previously.

For both the first and second aspects of the invention, the act of pouring the substance may be made safer and quicker by suitable dimensioning of the substance transferring device. Therefore, according to at least one embodiment of the invention, the substance is poured into an inlet of the substance transferring device which has a larger opening area than the inlet of the cell factory.

This enlargement of the fluid communications inlet interface is also expressed in a separate third aspect of the invention. According to this third aspect of the invention, a method is provided for introducing a substance into a multi-layered cell factory that has an inlet for receiving the substance, said inlet having a first opening area. The method comprises connecting to the inlet of the cell factory a substance transferring device which comprises an inlet having a second opening area which is larger than said first opening area, arranging the cell factory so that the layers of the cell factory extend substantially vertically, wherein said arranging is performed before, after or simultaneously with connecting the substance transferring device to the inlet of the cell factory, and pouring the substance into cell factory via the inlet of the substance transferring device.

The substance transferring device having a receiving inlet which is larger than the inlet of the cell factory may be regarded as an adapter which enlarges the opening and thereby facilitates pouring of the substance into the enlarged opening. Similarly to the previous aspects, the pouring according to the third aspect is suitably performed from an independent container.

Furthermore, in order to further facilitate the pouring of the substance, the dimensioning of the substance transferring device is suitable considered not only in relation to the cell factory inlet but also in relation to the source of pouring. Thus, within the scope of all of the above mentioned aspects, in accordance with at least one embodiment of the invention, in the case of pouring from an independent container, the act of pouring the substance comprises pouring the substance from an outlet of the independent container into an inlet of the substance transferring device that has a larger opening area than the opening area of said outlet.

Suitably, said independent container is a bottle, wherein the substance may be poured out from a top portion of the bottle. It should be noted that pouring from the top portion of the bottle generally means pouring from the top of a bottle neck. However, the opening may be located at other places of the top portion of an independent container. An advantage of having the opening at a top portion of a container, such as at the top of a bottle neck, is that the container does not need to be provided with a valve or seal for preventing the substance contained therein to flow out. This may be compared to the prior art intermediate large bottle shown in FIG. 1, in which a valve must be opened for drawing off the substance, instead of the simple pouring according to the present invention. It should further be noted that not only a bottle, but also any other type of container having a top portion from which the substance may be poured, presents a conceivable alternative.

It has been found advantageous to use a funnel for achieving said inlet of the substance transferring device which has a larger opening area than the inlet of the cell factory. It has also been found advantageous to use a funnel for achieving said inlet of the substance transferring device which has a larger opening area than the outlet of the independent container. A funnel enables both relations to be fulfilled, i.e. it can provide a larger opening area than both the inlet of the cell factory and the outlet of the independent container. Thus, according to at least one embodiment of the invention which is conceivable for all of the above aspects, the substance transferring device comprises a funnel, or similar utensil that is hollow and has an inlet opening which is larger or wider than its outlet opening, wherein the act of pouring or introducing the substance comprises pouring the substance into the funnel.

The outlet side of the funnel is suitably connectable to some type of conduit, such as a tube, that can be used to direct the substance into the inlet of the cell factory. In order to achieve positional variability and allow the vertically standing layers of the cell factory to receive the substance through a horizontally facing inlet of the cell factory, the conduit or at least a part of it is suitably movable relative to the inlet of the cell factory. This allows the funnel inlet to be facing substantially upwardly for receiving the substance to be poured. Alternatively, the funnel may be movable, e.g. pivotable, relative to the conduit. Thus, the flow communication inlet interface of the cell factory, which is initially represented by the inlet of the cell factory, may be redirected in various manners from facing substantially horizontally to facing substantially vertically once the funnel and conduit have been connected to the inlet whereby the inlet of the funnel obtains the function of said interface. However, it is currently considered to be advantageous and simple to use a flexible and bendable tube connected at one of its ends to the funnel and its other end being connected to the cell factory. The end of the tube connected to the cell factory may, if required, include or be attached to an adaptor which fits the inlet of the cell factory.

An advantage of using a flexible tube, apart from its benefits during the actual seeding and/or harvesting procedure, is that it may be folded so as to require only little space when being autoclaved. The folded tube may suitably be connected to the funnel before both of them are autoclaved.

It should be understood that as an alternative to using a separate conduit, such as a flexible tube, which is connectable to the funnel, the funnel and conduit may be formed in one piece. In such case the funnel and conduit are suitably made of the same material, e.g. a plastic material. If the funnel and conduit are made as separate pieces they may be formed from different materials, e.g. a glass funnel connectable to a plastic or silicone conduit.

From the above, it should be clear that the inventive combination of an enlarged receiving opening and a conduit, wherein at least one of them is movable relative to an inlet of a multi-layered cell factory, enables an operator to easily pour a substance into the cell factory via said enlarged receiving opening and conduit. In the case of a funnel connected to a flexible tube, additional advantages are obtainable. An advantage is that due to its flexibility and compactness, it may be packaged as a unit with the cell factory.

Harvesting out the cells from a cell factory is more complex than putting cells in a cell factory. Therefore, another advantage, which is applicable to all kinds of substance transferring devices encompassed by the invention, is that it is easy to pour in large quantities of different kinds of liquids without having to juggle a large bottle in the hood at the same time. Thus, after pouring the cell suspension into the cell factory, other reagents may be added using the same substance transferring device without removing it or having to add reagents to a large bottle. A further advantage is that the relatively small size of the substance transferring device, for example a funnel attached to a tube, does not compromise the airflow in the LAF bench.

According to at least one embodiment of the invention, in the case of the substance transferring device comprising a funnel, a holder arrangement is provided for holding the funnel. The funnel may be arranged at a desired height above the inlet of the cell factory by adjusting the holder arrangement to which the funnel is mountable. The holder arrangement may comprise any suitable height adjustable mechanism, such as e.g. a vertical stand on which an arm is displaceable up and down. The arm may comprise a claw or ring or other means for holding the funnel. Instead of using a vertical stand, it would be conceivable to mount the funnel to the side of the LAF bench at a desired height. The use of a height-adjustable holder arrangement is not limited to holding a funnel but could also be applicable to any substance transferring device encompassed by the present invention, thereby allowing the height, i.e. the vertical level, of the substance-receiving inlet of the substance transferring device to be controlled.

Standard cell factories generally contain two openings that can function identically. If the substance transferring device is held firmly, it can remain connected to one of the openings of the cell factory throughout the entire harvesting procedure. The other opening of the cell factory can then be used for pouring off substances while the opening with the connected substance transferring device may be used for adding new reagents.

The use of two openings or communication ports on the cell factory, one as an inlet and the other as an outlet, enables an effective and rapid handling of the entire seeding and harvesting procedure. Thus, according to at least one embodiment of the invention, not limited to the funnel and tube embodiments, the material contained in the cell factory can be removed through an outlet of the cell factory which is separate from an inlet of the cell factory, before any substance is introduced into the cell factory. In this case, said material may be e.g. the media in which the cells were suspended when seeded into the cell factory, rinsing substances for washing the cells, enzymes for detaching cells, etc. In the advantageous case when the substance transferring device is flexibly connected to the cell factory, the flexibility of the connection between the opening of the substance transferring device and the factory allows for removing of substances without disturbing the opening of the substance transferring device.

From the above it should now be clear that the substance transferring device according to any aspect of the invention may suitably be used for both seeding cells into the cell factory and for inserting other reagents, such as rinsing solutions or dissociation enzymes, as part of harvesting the cells from the cell factory.

Thus, according to at least one embodiment of the invention, the substance comprises a cell suspension for seeding cells in the cell factory. The cell suspension comprises cells suspended in media. Examples of commercially available media include DMEM and MEM among many others. Generally, mammalian cells are cultivated in cell factories, however it may be possible to cultivate other types of cells in cell factories as well.

Suitably, the internal volume of the substance transferring device is smaller than the total volume of the cell suspension to be seeded in the cell factory. This is possible since the substance transferring device does not need to retain any substance, but on the contrary to pass the substance directly into the cell factory. The substance transferring device, such as a combined funnel and tube, may thus be regarded as an extension of the inlet of the cell factory. Said small internal volume is particularly relevant in relation to the amount of cell suspension, since the cell suspension usually has a considerably larger volume than the other substances which may be poured into the cell factory through the substance transferring device. However, if desired, a substance transferring device may be selected so that its internal volume is smaller than any of the other substances used in the seeding and harvesting procedures.

As already mentioned, the other substances that may be poured through the substance transferring device comprises rinsing substances, dissociation substances and additional media for suspending the seeded, rinsed or detached cells, i.e. any liquid involved in culturing cells. Thus, according to at least one embodiment of the invention, said substance comprises a rinsing substance, such as Phosphate Buffered Saline (PBS), for rinsing cells that have been seeded into the cell factory, or dissociation enzymes, such as Accutase™ or Trypsin, for loosening adhered cells, or media for feeding loosened cells and stopping any enzymatic actions of dissociation enzymes. If desired, after introduction of the dissociation enzymes, the cell factory with the cells and dissociation enzymes may be incubated in an incubator. Suitably, the substance transferring device is disconnected from the cell factory before the incubation, and is reconnected after the incubation to allow media to be poured in. The disconnected part of the substance transferring device, such as an end of a tube, is suitably held in a contact-free manner in a LAF bench in order to avoid contamination, thereby allowing re-connection. In the case of a tube, a portion of a tube may be attached, e.g. taped, to an adjacent structure such as a vertical stand or arm included in the previously described holder arrangement, while keeping the tube end from contacting said structure or any other nearby structure. As an alternative, another substance transferring device may be used after the incubation.

It should be noted that all aspects of the present invention are applicable to introducing a substance into a multi-layered cell factory. Even though, for the sake of simplicity, the background description has been kept to a ten-layered cell factory, the present invention is also applicable to multi-layered cell factories having other numbers of layers. For instance, there are currently commercially available factories having one, two, four, ten or forty layers. The invention would be applicable to any one of these alternatives, as well as any future number of layers within that range or even above that range, such as e.g. fifty layers.

A method of introducing a substance into a multi-layered cell factory has now been described in accordance with at least three aspects of the invention. Additionally there is also a fourth aspect of the invention related to a cell-cultivation system. Thus, according to the fourth aspect of the invention, a cell-cultivation system is provided. The system comprises a multi-layered cell factory having an inlet for receiving a substance. The system also comprises a substance transferring device having a first end which is connectable to the inlet of the cell factory, and a second end having an inlet for receiving the substance. The received substance is transferred directly into the cell factory. The inlet of the substance transferring device has a larger opening area than the inlet of the cell factory, and said second end is movable relative to said first end for enabling said first end to be arranged at a non-zero angle relative to said second end.

As previously discussed, the inlet of the substance transferring device may suitably face vertically (upwardly) while the inlet to the cell factory may face horizontally (laterally). Thus, said non-zero angle may advantageously be about 90° when the substance is introduced into the cell factory via the substance transferring device. However, other non-zero angles are also conceivable.

The cell-cultivation system may suitably also comprise an independent container from which the substance may be poured. The cell-cultivation system may also comprise a height-adjustable holder arrangement. The independent container, the substance transferring device and the holder arrangement may have any one of the previously described features. Thus, the fourth aspect of the invention encompasses any embodiments or any features described in connection with the previously described aspects of the invention, as long as those embodiments or features are compatible with said cell-cultivation system.

According to a fifth aspect of the invention, a method of cultivating cells in a multi-layered cell factory is provided. The method according to the fifth aspect includes introducing a substance in a similar manner to the previously described aspects and should, therefore, be understood to encompass any embodiments or any features described in connection with the previously described aspects of the invention, as long as those embodiments or features are compatible with this method. The method according to the fifth aspect comprises
connecting a substance transferring device to an inlet of the cell factory,
arranging the cell factory so that the layers of the cell factory extend substantially vertically, wherein the connecting and arranging may be performed in either order,
pouring a cell suspension through the substance transferring device which directly transfers the media and cells into the cell factory, and
pouring off the media from the cell factory, suitably with the substance transferring device remaining connected to the inlet of the cell factory.

This method encompasses situations where analysis of the media is of interest, and wherein the cells are not required to be poured off. However, this method also encompasses situations in which the cells are suspended in the poured-off media. The method also encompasses use of any other substance, such as a rinsing substance or a dissociation substance, as described below.

Thus, the method according to the fifth aspect may optionally also comprise
pouring a rinsing substance, such as Phosphate Buffered Saline (PBS), into the cell factory via the substance transferring device in order to rinse the layers, and
pouring off the rinsing substance from the cell factory.

Generally, it is desirable to rinse the residual media from the cells, however in some cases it may not be necessary, and therefore the above steps of using rinsing substance are optional.

Furthermore, the method according to the fifth aspect may optionally also comprise
pouring dissociation enzymes, such as Accutase™ or Trypsin, into the cell factory via the substance transferring device in order to loosen the cells from the layer walls,
disconnecting the substance transferring device,
incubating the factory with the cells and dissociation enzymes in an incubator,
re-connecting the substance transferring device or alternatively connecting another substance transferring device to the inlet of the cell factory,
pouring media via the substance transferring device into the cell factory to stop the enzyme reaction and feed the cells, and
pouring off the media from the cell factory, suitably including therein suspended cells.

Generally, it is desirable to dissociate the cells with an enzyme. However, some cells may be detached without a dissociation enzyme and, therefore, the above steps of using a dissociation enzyme are optional.

From the above, it should now be clear that the present invention enables the operator to directly pour, via a substance transferring device, a substance into a multi-layered cell factory which is arranged with its layers standing up vertically, without needing an intermediate substance-retaining container. An advantageous embodiment is the combination of a tube with a funnel, however, as explained above other alternatives are also conceivable and envisaged by the present invention. In general terms, the substance transferring device may be regarded as having at least two portions. One portion is movable or pivotable relative to the other portion. This allows the substantially horizontally facing inlet to the vertically standing layers to be transformed or extended to a substantially upwardly facing inlet of the substance transferring device into which the substance may be poured.

In regard to the combination of a tube and a funnel, this is incorporated in a sixth aspect of the invention. According to this sixth aspect, a use of a funnel is provided. In particular, the use of a funnel connected to one end of a tube for introducing a substance into a multi-layered cell factory at the other end of the tube. The sixth aspect of the invention encompass any embodiments or any features described in connection with the previously described aspects of the invention, as long as those embodiments or features are compatible with using a funnel connected to a tube.

### BRIEF DESCRIPTION OF THE DRAWINGS

FIG. 1 illustrates schematically a method for seeding cells into a multi-layered cell factory according to the prior art.

FIGS. 2 and 3 illustrate schematically a cell-cultivation system in accordance with at least one embodiment of the present invention.

FIG. 4 illustrates schematically a method wherein an operator introduces a substance into a multi-layered cell factory in accordance with at least one embodiment of the invention.

### DETAILED DESCRIPTION OF THE DRAWINGS

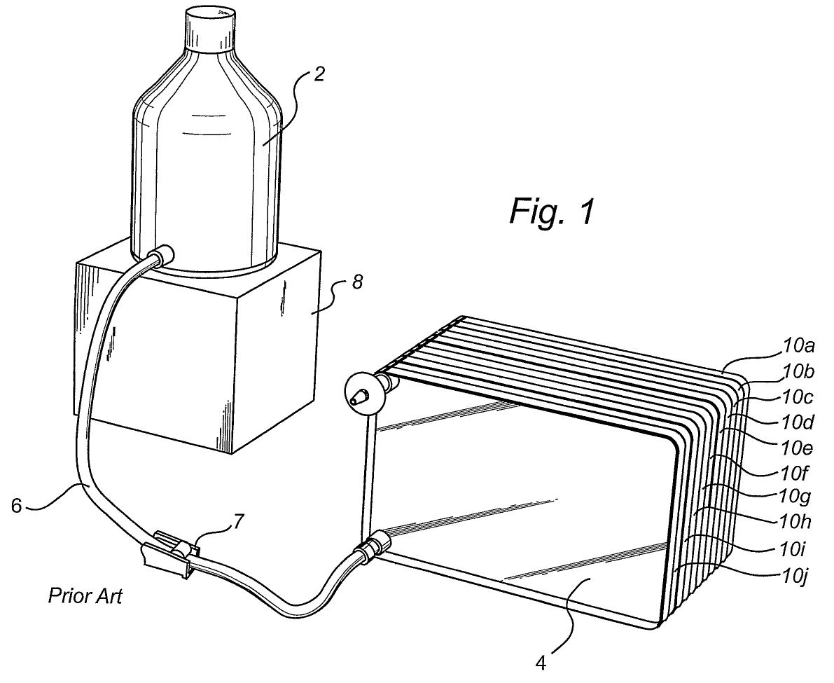

The prior art method illustrated in FIG. 1 has previously been discussed under the heading “Background of the invention” to which reference is made without any further discussion here.

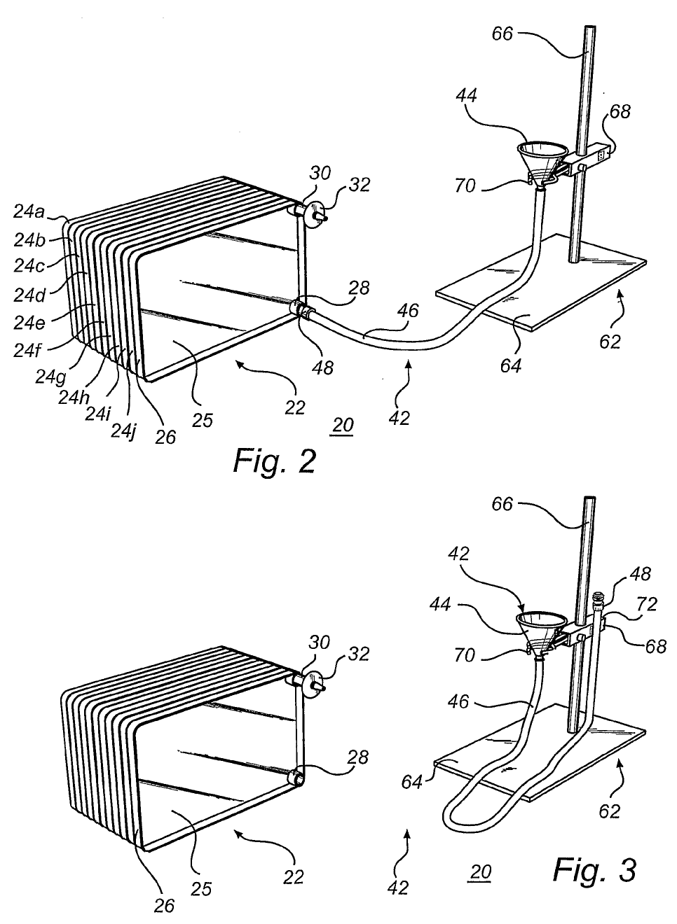

With reference to FIGS. 2 and 3, a cell-cultivation system 20 according to at least one embodiment of the present invention is schematically illustrated. The cell-cultivation system 20 comprises a multi-layered cell factory 22, a substance transferring device 42 which is herein illustrated as a funnel 44 connected to a tube 46, and a holder arrangement 62 for holding the substance transferring device 42.

The multi-layered cell factory 22 is herein illustrated as having ten layers 24a-24j, however other number of layers are also conceivable. Each layer 24a-24j has a fluid-receiving volume which is defined by two opposing, comparatively broad, plate-like rectangular wall sides 25 and four comparatively narrow strip-like wall sides 26 that interconnect the edge portions of said opposing rectangular wall sides. The rectangular geometry is merely an example and other geometries would also be conceivable. One or more channels (not illustrated) allow for the layers to be in fluid communication with each other.

The multi-layered cell factory is provided with a first and a second fluid communication port 28 and 30, respectively, both of which may be used either as an inlet or as an outlet. In the illustrated example, only the first fluid communication port 28 is used as an inlet. The second communication port 30 is provided with a plug 32 which is removable for using it as an outlet. Both ports 28, 30 are in fluid communication with said one or more channels, which in turn are in fluid communication with the layers 24a-24j. As illustrated in the figures, the layers 24a-24j of the cell factory 22 are standing vertically, which means that the geometrical plane defined by each one of said comparatively broad plate-like rectangular wall sides 25 is perpendicular to the horizontal ground on which the cell factory 22 rests. In this position of the multi-layered cell factory 22, the openings of the fluid communication ports 28, 30 are facing horizontally, i.e. they extend from the cell factory 22 along a line which is parallel to the horizontal ground. Thus, the direction of the flow through the first communication port 28, functioning as an inlet, will be a substantially horizontal flow into the cell factory 22.

The substance transferring device 42 is herein illustrated as a tube 46 connected to a funnel 44 into which a substance is to be poured, however, other alternatives would also be possible. For instance, instead of a funnel, an alternative would be to use a sheath or a slide having e.g. a U-shaped or V-shaped cross-section, wherein the slide is inclined downwards towards the cell factory or towards a connected tube, and wherein substance may be poured into the opening of the U or V and flow down the slide, either via a tube or directly into the cell factory. Another alternative would be to simply use a tube having one end connected to the inlet of the cell factory and the other end being provided with a slit for splitting the tube end open and thereby providing an area large enough for receiving a substance to be poured.

Turning now back to FIGS. 2 and 3, and in particular FIG. 2, wherein one of the end portions of the tube is provided with an adaptor 48 for connecting the tube to the first communication port 28 to be used as an inlet into the cell factory 22. The length of the tube 46 should be chosen so that on the one hand an effective flow of substance is obtainable, and on the other hand an easy handling of the cell factory 22 is allowable when it is turned or rotated so as to ensure that the introduced substance has reached all inner wall sides of the layers 24a-24j (e.g. for ensuring that cells from a cell suspension will become attached and substantially evenly distributed along all the inner wall sides). The funnel 44 is connected to the other end other of the tube 46. The funnel 44 is, in this example, in the form of a hollow cone with a pipe portion extending from the smaller end and into the tube. The funnel 44 is dimensioned and configured to catch a substance to be poured into it and direct the substance downwards into the tube 46. As can be seen from the figures, the large fluid-receiving opening of the funnel 44, i.e. the geometrical base of the inverted cone-shape, is facing upwardly and perpendicularly to the horizontal ground plane, and therefore also perpendicularly to the direction that the fluid communication ports 28, 30 face when the cell factory 22 is positioned with the layers 24a-24j standing vertically. It may also be noted that the opening area of the funnel 44, i.e. the area of the geometrical base of the inverted cone-shape, is considerably larger than the opening area of the fluid communication ports 28, 30.

The funnel 44 is held at a vertical level above the inlet 28 of the cell factory 22 by means of the holder arrangement 62, thereby enabling the gravitational force to favourably affect the flow of the substance from the funnel 44, via the tube 46, and into the cell factory 22. The holder arrangement 62 is herein illustrated as a stand which comprises a stabilizing base plate 64 from which a vertical guiding rod 66 extends. A horizontally extending arm 68 comprising a gripping end 70 is displaceable up and down the vertical guiding rod 66, whereby the vertical level of the gripping end 70 is adjustable. The funnel 44 is held by the gripping end 70 of the arm 68, whereby the height of the funnel 44 relative to the inlet 28 of the cell factory 22 is adjustable.

FIG. 3 illustrates how the tube 46 may suitably be attached to the holder arrangement 62 without risking contamination of the tube end to be re-connected to the cell factory 22, e.g. after the cell factory 22 and its contents have been incubated. In this figure, it is illustrated that the tube 46 has been taped to a portion 72 of the arm 68 which is located on the other side of the guiding rod 66 opposite the gripping end 70 side. Note that the attachment point of the tube 46 is somewhat spaced from the tube end, so as to allow the tube end to be free from any contact until it is re-connected to the cell factory 22.

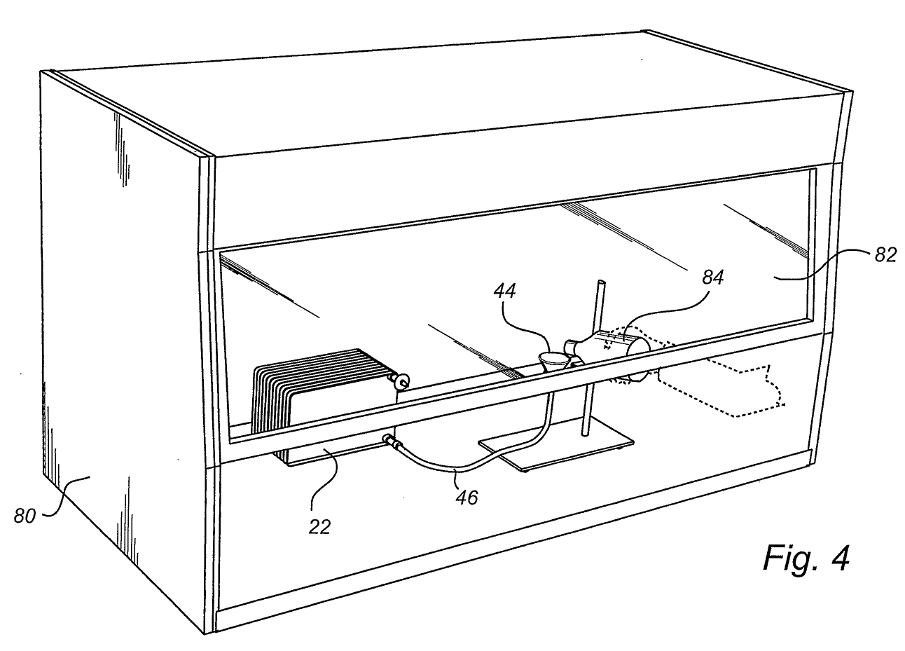

FIG. 4 illustrates schematically a method wherein an operator introduces a substance into a multi-layered cell factory 22 in accordance with at least one embodiment of the invention. The cell-cultivation system illustrated in FIGS. 2 and 3 is now placed inside a standard LAF bench 80. To set up the cell-cultivation system may take about five minutes, and pouring e.g. 1500 ml of cell suspension into the cell factory 22 via the funnel 44 and tube 46 may also take about five minutes. This may be compared to the prior art methods which typically take at least 20 minutes for introducing the same amount into the cell factory 22. As is shown in the figure, an operator can easily stick his or her arm under the hood 82 of the LAF bench 80 in order to pour the substance from an independent bottle 84 into the funnel 44. Depending on the volume of the substance to be introduced into the cell factory 22, different numbers of bottles may be used. For instance, 1500 ml of cell suspension is suitably divided into three bottles as is customary when initially harvesting the cells from regular tissue culture flasks. When the operator has emptied the first bottle he or she easily takes the next bottle and pours its contents down the funnel, and so on. For a rinsing or dissociation substance, it is usually enough to use a single bottle or the like. It should be noted that the funnel may be considerably smaller than the volume of the substance to be introduced into the cell factory. This is because the funnel merely functions to capture and direct the poured substance instead of containing it. Therefore, the funnel can be placed at a height which gives an effective flow and which is still convenient for an operator to pour from, despite the shielding hood of the LAF bench.

From the above it should be clear that the present invention provides a quick introduction of substance into a multi-layered cell factory which results in an effective and even distribution of substance over all the layers. This is in particular advantageous when it comes to seeding cells, since they should preferably be evenly attached and distributed in all the layers. It should also be clear that the entire procedure is facilitated since the same substance transferring device may be used for all substances to be introduced into the multi-layered cell factory. In fact, the substance transferring device may stay connected to the cell factory while a substance is removed from the cell factory so as to allow a new substance to be subsequently introduced without any loss of valuable time.

1. A method of introducing a substance into a multi-layered cell factory, comprising
connecting a substance transferring device to an inlet of the cell factory,
arranging the cell factory so that the layers of the cell factory extend substantially vertically, wherein said arranging is performed before, after or simultaneously with connecting the substance transferring device to the inlet of the cell factory, and
pouring the substance from an independent container into the cell factory via the substance transferring device which directly transfers the substance into the cell factory.

2. A method of introducing a substance into a multi-layered cell factory that has an inlet for receiving the substance, comprising
connecting to the inlet of the cell factory a substance transferring device which comprises its own inlet for receiving the substance and directly transferring the substance to the inlet of the cell factory, and
pouring the substance into the inlet of the substance transferring device when the opening area of the inlet of the substance transferring device faces another direction than the opening area of the inlet of the cell factory.

3. The method as claimed in claim 1, wherein the act of pouring the substance comprises pouring the substance into an inlet of the substance transferring device which has a larger opening area than the inlet of the cell factory.

4. A method of introducing a substance into a multi-layered cell factory that has an inlet for receiving the substance, said inlet having a first opening area, comprising
connecting to the inlet of the cell factory a substance transferring device which comprises an inlet having a second opening area which is larger than said first opening area,
arranging the cell factory so that the layers of the cell factory extend substantially vertically, wherein said arranging is performed before, after or simultaneously with connecting the substance transferring device to the inset of the cell factory, and
pouring the substance into cell factory via the inlet of the substance transferring device.

5. The method as claimed in claim 2, wherein the act of pouring the substance comprises pouring the substance from an independent container into the substance transferring device which directly transfers the substance into the cell factory.

6. The method as claimed in claim 1, wherein the act of pouring the substance from an independent container comprises pouring the substance from an outlet of the independent container into an inlet of the substance transferring device that has a larger opening area than the opening area of said outlet.

7. The method as claimed in claim 6, wherein said independent container is a bottle, wherein the act of pouring the substance from an outlet of the independent container comprises pouring the substance out from a top portion of the bottle.

8. The method as claimed in claim 1, wherein said substance transferring device comprises a funnel, wherein the act of pouring or introducing the substance comprises pouring the substance into the funnel.

9. The method as claimed in claim 8, wherein said substance transferring device comprises a tube connected at one of its ends to the funnel, wherein the act of connecting the substance transferring device comprises connecting the other end of the tube to the inlet of the cell factory.

10. The method as claimed in claim 8, comprising adjusting a holder arrangement for holding the funnel at a selected height above the inlet of the cell factory, and mounting the funnel to the holder arrangement so that the funnel is located at the selected height, wherein the acts of adjusting and mounting are performable in either order.

11. The method as claimed in claim 1, wherein said substance comprises a cell suspension for the seeding of cells in the cell factory.

12. The method as claimed in claim 11, wherein the internal volume of the substance transferring device is smaller thin the total volume of the cell suspension to be seeded in the cell factory.

13. The method as claimed in claim 1, wherein said substance comprises a rinsing substance, such as Phosphate Buffered Saline (PBS), for rinsing cells that have been seeded into the cell factory.

14. The method as claimed in claim 1, wherein said substance comprises dissociation enzymes, such as Accutase™ or Trypsin, for loosening seeded cells from the layer walls.

15. The method as claimed in claim 13, comprising, before introducing said substance into thy cell factory, removing other material contained in the cell factory through an outlet of the cell factory which is separate from the inlet of the cell factory.

16. The method as claimed in claim 15, wherein said substance transferring device is kept connected to the inlet of the cell factory during the act of removing said other material.

17. A cell-cultivation system, comprising
a multi-layered cell factory having an inlet for receiving a substance, and
a substance transferring device having
a first end which is connectable to the inlet of the cell factory, and
a second end having an inlet for receiving the substance, whereby the received substance is transferable directly into the cell factory,
wherein the inlet of the substance transferring device has a larger opening area than the inlet of the cell factory, and wherein said second end is movable relative to said first end for enabling said first end to be arranged at a non-zero angle relative to said second end.

18. The system as claimed in claim 17, wherein said substance transferring device comprises a funnel, wherein the inlet of the substance transferring device is the wider opening of the funnel.

19. The system as claimed in claim 18, wherein said substance transferring device comprises a tube which is connectable at one of its ends to the funnel, wherein the other end of the tube is said first end which is connectable to the inlet of the cell factory.

20. The system as claimed in claim 18, comprising a height-adjustable holder arrangement for holding the funnel at a selected height above the inlet of the cell factory.

21. The system as claimed in claim 17, comprising an independent container having an outlet from which the substance is pourable into the substance transferring device for direct transfer into the cell factory.

22. The system as claimed in claim 21, wherein the inlet of the substance transferring device has a larger opening area than the opening area of said outlet of the independent container.

23. The system as claimed in claim 22, wherein said independent container is a bottle and wherein the outlet of the independent container is located at a top portion of the bottle.

24. The system as claimed in claim 17, wherein said cell factory comprises an outlet, which is separate from the inlet of the cell factory, for enabling substance to be removed from the cell factory while the substance transferring device is connected to the inlet of the cell factory.

25. A method of cultivating cells in a multi-layered cell factory, comprising
connecting a substance transferring device to an inlet of the cell factory,
arranging the cell factory so that the layers of the cell factory extend substantially vertically, wherein the connecting and arranging are performable in either order,
pouring a cell suspension through the substance transferring device which directly transfers the media and cells into the cell factory, and
pouring off the media from the cell factory.

26. The method as claimed in claim 25, comprising
pouring a rinsing substance, such as Phosphate Buffered Saline (PBS), into the cell factory via the substance transferring device in order to rinse the layers, and
pouring off the rinsing substance from the cell factory.

27. The method as claimed in claim 25, comprising
pouring dissociation enzymes, such as Accutase™ or Trypsin, into the cell factory via the substance transferring device in order to loosen the cells from the layer walls,
disconnecting the substance transferring device,
incubating the factory with the cells and dissociation enzymes in an incubator,
re-connecting the substance transferring device, or alternatively, connecting another substance transferring device to the inlet of the cell factory,
pouring media via the substance transferring device into the cell factory to stop the enzyme reaction and feed the cells, and
pouring off the media from the cell factory, suitably including therein suspended cells.

28. The method as claimed in claim 25, wherein said substance transferring device is kept connected to the inlet of the cell factory during the act of pouring off the rinsing substance or media from the cell factory.

29. A use of a funnel connected to one end of a tube for introducing a substance into a multi-layered cell factory at the other end of the tube.

30. The use as claimed in claim 29, comprising performing the acts defined in the method according to claim 1.

31. The use as claimed in claim 29, wherein said funnel, tube and multi-layered cell factory form part of a cell-cultivation system according to claim 17.
